SEQUENCE LISTING (1 sequence)

SEQ ID NO: 1 - DNA, 25 bp, Artificial
  morpholino antisense oligonucleotide PMOE23
ggccaaacctcggcttacctgaaat

(19) United States
(12) Patent Application Publication
(10) Pub. No.: US 2017/0173069 A1
(43) Pub. Date: Jun. 22, 2017

(54) METHODS OF NUCLEOTIDE AND GENE DELIVERY USING MEMBRANE TRANSPORT MECHANISMS

(71) Applicant: The Charlotte Mecklenburg Hospital Authority d/b/a Carolinas HealthCare System, Charlotte, NC (US)

(72) Inventor: Qi Long Lu, Charlotte, NC (US)

(21) Appl. No.: 15/379,997

(22) Filed: Dec. 15, 2016

Related U.S. Application Data
(60) Provisional application No. 62/269,516, filed on Dec. 18, 2015.

(51) Int. Cl. A61K 31/7125; A61K 38/28; A61K 31/451; A61K 31/712

(52) U.S. Cl. CPC A61K 31/7125; A61K 31/712; A61K 38/28; A61K 31/451; A61K 2121/00; A61K 2300/00

(57) ABSTRACT

The present invention provides methods of delivering a nucleic acid to a cell or tissue, the method including administering the nucleic acid in combination with at least one drug that modulates a membrane transport mechanism. Methods of treating various diseases are also provided.

### STATEMENT OF PRIORITY

This application claims the benefit of U.S. Provisional Application Ser. No. 62/269,516, filed Dec. 18, 2015, the disclosure of which is incorporated by reference in its entirety.

### STATEMENT REGARDING ELECTRONIC FILING OF A SEQUENCE LISTING

A Sequence Listing in ASCII text format, submitted under 37 C.F.R. §1.821, entitled 1184-18_ST25.txt, 613 bytes in size, generated on Feb. 21, 2017 and filed via EFS-Web, is provided in lieu of a paper copy. This Sequence Listing is hereby incorporated herein by reference into the specification for its disclosures.

### RESERVATION OF COPYRIGHT

A portion of the disclosure of this patent document contains material which is subject to copyright protection. The copyright owner, The Charlotte-Mecklenburg Hospital Authority, doing business as “Carolinas HealthCare System,” Charlotte, N.C., has no objection to the reproduction by anyone of the patent document or the patent disclosure, as it appears in the U.S. Patent and Trademark Office patent file or records, but otherwise reserves all copyright rights whatsoever.

### FIELD OF THE INVENTION

The present invention relates to delivery of genetic material into cells and tissues. The invention further relates to methods of treating various diseases and disorders by delivering genetic material to a subject in need thereof.

### BACKGROUND OF THE INVENTION

It has been realized for more than 30 years that the biggest obstacle for successful application of gene and nucleotide therapy is the difficulty in delivery. There have been many old methods for enhancing delivery of nucleic acids-based agents. The old methods can be divided roughly into 3 classes, physical, biochemical and viral methods. Physical methods, such as ultrasound, microwave and electroporation aim to create micro sized pores on cell membrane and force nucleic acids cargo through cell membrane into target cells. Biochemical methods used polymers and nanoparticles, such as pluronics, cationic polymers, polypeptides, polyethylene glycols (PEGS) and liposomes, with the aim to wrap and pack the cargo nucleic acids (nucleotides and genes) for more efficient entry into target cells. Many different viruses have also been tried for gene delivery and for expression of oligonucleotides. These methods have so far been largely limited to the uses in cell culture and ex-vivo. All of these huddles have prevented the nucleic acid based experimental therapies from achieving their therapeutic potential in vivo in clinic. The safety, especially long-term safety of the use of viruses in human remains a biggest concern.

### SUMMARY OF THE INVENTION

The present invention is based in part on the surprising finding that the seemingly unrelated insulin release and activation of the fatty acid transporter and/or glucose transporter pathway can enhance the delivery of nucleotide sequences.

Provided herein according to some embodiments of the present invention are methods of delivering genetic material, such as a nucleic acid, to a cell or tissue, the method including administering the genetic material in combination with at least one drug that modulates a membrane transport mechanism.

Also provided herein are methods of treating cancer, an infectious disease, diabetes or a genetic disorder, the method including administering to a subject in need thereof a therapeutically effective amount of a genetic material as described above in combination with at least one drug that modulates a membrane transport mechanism.

Further provided herein is a transformed cell produced by a process of delivering a genetic material, the process including administering the genetic material in combination with at least one drug that modulates a membrane transport mechanism.

Still further provided is a transgenic animal produced by the process of delivering a genetic material, the process including administering the genetic material in combination with at least one drug that modulates a membrane transport mechanism.

These and other embodiments of the invention are set forth in more detail in the description of the invention below.

### BRIEF DESCRIPTION OF THE DRAWINGS

FIG. 1 shows a comparison of mouse treated PMO only and PMO with Repaglinide. The mdx dystrophic mice containing a nonsense mutation in the dystrophin exon 23 are treated with antisense oligonuleotide PMOE23 (5′GGCCAAACCTCGGCTTACCTGAAAT-3′) (SEQ ID NO:1) via intravenous injection and the muscles are examined 2 weeks later.

FIG. 2 shows a comparison of levels of dystrophin expression after treatment with morpholino antisense oligonucleotide PMOE23 and Eicosapentaenoic acid (EPA)-linked PMOE23 in 4-6 weeks old mdx dystrophic mice.

FIG. 3 shows a comparison of levels of dystrophin expression after treatment with morpholino antisense oligonucleotide PMOE23 and PMOE23 in combination with the use of insulin in 4-6 weeks old mdx dystrophic mice.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. §1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols, in Molecular Biology (Green Publishing Associates, Inc. and John Wiley & Sons, Inc., New York).

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity, GC content and the like, refers to variations of ±20%, ±10%, ±5%, ±1%, ±0.5%, or even ±0.1% of the specified amount.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

The transitional phrase “consisting essentially of” means that the scope of a claim is to be interpreted to encompass the specified materials or steps recited in the claim, “and those that do not materially affect the basic and novel characteristic(s)” of the claimed invention.

The term “consists essentially of” (and grammatical variants), as applied to a nucleotide, polynucleotide or polypeptide sequence related to this invention, means a nucleotide, polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to express the encoded polypeptide of at least about 50% or more as compared to the expression level of a polynucleotide consisting of the recited sequence.

The term “modulate,” “modulates” or “modulation” refers to enhancement (e.g., an increase) or inhibition (e.g., a reduction) in the specified activity. Accordingly, a modulator may be an activator, a stimulator or an inhibitor.

The term “enhance” or “increase” or grammatical variations thereof as used herein refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold or an increase of at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 225%, 250%, 275%, 300%, 400%, 500% or more.

“Drug” as used herein refers to a chemical entity or biological product, or combination of chemical entities or biological products, administered to a person to treat, prevent, or delay the onset or control (e.g., delay the progression), of a disease or condition. The term “drug” as used herein is synonymous with the terms “medicine,” “medicament,” “therapeutic intervention,” or “pharmaceutical product.” In some embodiments, the drug may be approved by a government agency for treatment of at least one specific disease or condition.

As used herein, “effective amount” refers to an amount of a compound or composition that is sufficient to produce a desired effect, which can be a therapeutic effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating a disease or disorder by the methods of the invention can be determined by detecting a clinical improvement as indicated by a change in the subject's symptoms and/or clinical parameters as would be well known to one of skill in the art.

By the terms “treat,” “treating,” or “treatment,” it is intended that the severity of the subject's condition is reduced or at least partially improved or modified and that some alleviation, mitigation or decrease in at least one clinical symptom is achieved.

The terms “prevent,” “preventing,” and “prevention” (and grammatical variations thereof) refer to a decrease or delay in the extent or severity of a disease, disorder and/or clinical symptom(s) after onset relative to what would occur in the absence of carrying out the methods of the invention prior to the onset of the disease, disorder and/or clinical symptom(s).

“Genetic material” as used herein means all forms of DNA and RNA as well as modified and non-modified nucleotides.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides) or produced by cell biology techniques commonly used for vector production. Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid may include some or all of the 5′ non-coding (e.g., promoter) sequences that are immediately contiguous to a coding sequence. The term therefore includes, for example, a recombinant DNA/synthetic polynucleotide that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA/synthetic polynucleotide that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The terms “exogenous” and/or “heterologous” as used herein can include a nucleotide sequence that is not naturally occurring in the nucleic acid construct and/or delivery vector (e.g., virus delivery vector) in which it is contained and can also include a nucleotide sequence that is placed into a non-naturally occurring environment and/or position relative to other nucleotide sequences (e.g., by association with a promoter or coding sequence with which it is not naturally associated). A heterologous or exogenous nucleotide sequence or amino acid sequence of this invention can be any heterologous nucleotide sequence and/or amino acid sequence that has been introduced into a cell and can include a nucleotide sequence and/or amino acid sequence for which an original version is already present in the cell and the heterologous nucleotide sequence and/or amino acid sequence is a duplicate of the original naturally occurring version, and/or the heterologous nucleotide sequence or amino acid sequence can be introduced into a cell that does not naturally comprise the same nucleotide sequence and/or amino acid sequence.

The term “synthetic polynucleotide” refers to a polynucleotide sequence that does not exist in nature but instead is made by the hand of man, either chemically, or biologically (i.e., in vitro modified) using cloning and vector propagation techniques.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence, means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

A “promoter” is a nucleotide sequence that controls or regulates the transcription of a nucleotide sequence (i.e., a coding sequence) that is operably associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

Embodiments of the present invention are directed to enhanced delivery (or uptake) of genetic material to a cell or tissue utilizing a drug that modulates a membrane transport system. The delivery may be in vitro or in vivo delivery.

In some embodiments, the present invention provides methods of delivering a nucleic acid and/or a vector to a cell or tissue, the method comprising, consisting essentially of, or consisting of administering the nucleic acid and/or vector in combination with at least one drug that modulates a membrane transport mechanism. Embodiments of the invention can be further utilized for the delivery of nucleic acids, such as oligonucleotides, e.g., antisense oligonucleotides, siRNA, gapmers, and microRNA mimics. The nucleotides, oligonucleotides and long chain nucleotides can be of any chemical modification, such as 2′-O-methyl phosphorothioate, 2′-O-methoxyethyl (MOE), Locked/bridged nucleic acids (LNA/BNA), Phosphoramidate, 2′-O-aminopropyl phosphodiester ribonucleotides, Phosphorodiamidate Morpholino Oligonucleotides (PMO), Peptide nucleic acid (PNA), Hexitol nucleic acid (HNA) and tricyclo-DNA (tcDNA), with and without any further modification. The nucleic acid may be an oligonucleotide from 10mer to 500mer. The enhanced delivery of plasmid or gene expression vectors are to be used for any purpose of gene therapy.

A “nucleic acid” may be, but is not limited to, any oligonucleotide or a long chain oligonucleotide (up to, e.g., about 200 basepairs), as well as expression vectors, such as plasmid or synthetic expression sequences. In some embodiments, the nucleic acid may be an oligonucleotide or a long chain oligonucleotide, such as an antisense oligonucleotide for, in some embodiments, splicing modification (exon skipping, alternative use of promoters and exons, gene knockdown), an siRNA or a gapmer for, in some embodiments, gene silencing, or a microRNA (miRNA) or a miRNA mimic for, in some embodiments, RNA silencing and gene expression regulation, and for gene delivery.

The method of preparing such oligonucleotides or long chain oligonucleotides, as well method of chemically modifying such oligonucleotides or long chain oligonucleotides is not particularly limited, and any method to prepare or to synthesize such oligonucleotides or long chain oligonucleotides, and to chemically modify such oligonucleotides or long chain oligonucleotides, may be used that would be appreciated and understood by one of skill in the art.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmids, synthetic vectors, PCR-synthesized vectors with or without complexation with liposomes, lipids (cytofectins), protein and peptide, viral vectors (e.g., retrovirus, lentivirus, adeno-associated virus, poxvirus, alphavirus, baculovirus, vaccinia virus, herpes virus, Epstein-Barr virus, and adenovirus vectors). The term “vector” includes both viral and nonviral (e.g., plasmid) nucleic acid molecules for introducing a nucleic acid into a cell in vitro, ex vivo, and/or in vivo. In some embodiments, the heterologous nucleic acid that is expressed by the vector is an oligonucleotide or a long chain oligonucleotide that modulates a membrane transport mechanism.

It will be apparent to those skilled in the art that any suitable vector can be used to deliver a heterologous nucleic acid of this invention. The choice of delivery vector can be made based on a number of factors known in the art, including age and species of the target host, in vitro vs. in vivo delivery, level and persistence of expression desired, intended purpose (e.g., for therapy or polypeptide production), the target cell or organ, route of delivery, size of the isolated nucleic acid, safety concerns, and the like.

Suitable vectors also include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., one, two, three, four, five or more heterologous nucleotide sequences.

This invention uses a membrane transport mechanism, i.e., a facilitated transport (translocation, movement, etc.) of an agent across plasma membranes, e.g., across membrane bilayers. In particular embodiments, the mechanism is fatty acid transport. In other embodiments, the mechanism involves glucose transport. In particular, the fatty acid and/or glucose transporter pathways may be activated and/or modified for cargo nucleic acid delivery and is used to modify cargo nucleic acid for enhanced delivery. That is, in addition, a fatty acid moiety or a sugar moiety may be linked (in some instances, directly, i.e., covalently) to the nucleic acid and/or vector. The fatty acid may be saturated or unsaturated. The fatty acid can be a small, medium chain, long chain or very long chain fatty acid. In some embodiments, the fatty acid is a medium chain, long chain or very long chain fatty acid. In some embodiments, the fatty acid has a chain length from 5 to 50.

In general, drugs that increase blood insulin levels can be used in the methods of the present invention. Accordingly, at least one drug selected from the following non-limiting examples may be used: a sulfonylurea, a biguanide, a meglitinide, a thiazolidinedione, a DPP-4 modulator, a SGLT2 modulator, an alpha-glucosidase modulator, and a bile acid sequestrant. Examples of such drugs include, but are not limited to, metformin (glucophage, glumetza); sulfonylureas (glyburide, glipizide and glimepiride); Meglitinides (repaglinide and nateglinide); thiazolidinediones (rosiglitazone and pioglitazone); DPP-4 modulators (sitagliptin, saxagliptin and linagliptin); GLP-1 receptor agonists (exenatide and liraglutide); SGLT2 modulators (canagliflozin and dapagliflozin); insulin, dipyridamole, oligomycin and a K⁺_{ATP} channel modulator. The drugs may be used in combination. In some embodiments, the drug is insulin and/or repaglidine.

The nucleic acid and/or vector may be administered before the drug, after the drug, or the same time as the drug. The nucleic acid and/or vector may be administered in a composition including the drug. Exemplary modes of administration include oral, rectal, transmucosal, topical, intranasal, inhalation (e.g., via an aerosol), buccal (e.g., sublingual), vaginal, intrathecal, intraocular, in utero (or in ovo) and parenteral. In particular embodiments, the nucleic acid and/or vector may be administered parenterally. In some embodiments, the nucleic acid and/or vector may be administered intraperitoneally, intravenously, subcutaneously, intradermally, intramuscularly [including administration to skeletal, diaphragm and/or cardiac muscle], intrapleurally, intracerebrally, and intraarticularly), topically (e.g., to both skin and mucosal surfaces, including airway surfaces, and transdermal administration), and the like, as well as direct tissue or organ injection (e.g., to liver, skeletal muscle, cardiac muscle, diaphragm muscle or brain). In some embodiments, the nucleic acid and/or vector and drug are administered at the same site. In other embodiments, the nucleic acid and/or vector and drug are administered at different sites. The most suitable route in any given case will depend on the nature and severity of the condition being treated and on the nature of the particular nucleic acid, vector and/or drug that is being used.

Embodiments of the present invention further provide methods of treating cancer, an infectious disease, diabetes or a genetic disorder, the method comprising, consisting essentially of or consisting of administering to a subject in need thereof a nucleic acid and/or a vector in combination with at least one drug that modulates a membrane transport mechanism.

“Infectious disease” as used herein refers generally to microbial (e.g., bacterial, fungal, viral) and/or parasitic infections and include, but are not limited to: Skin and soft tissue infections such as: folliculitis, furuncles, and carbuncles; erysipelas; lymphangitis; cellulitis, necrotizing soft tissue infections, diabetic foot infections, decubitus ulcers; Bone and joint infections such as: osteomyelitis, infectious arthritis, mastoiditis; Central nervous system infections arising from meningococcal, pneumococcal, diplococcal, H. influenzae, as well as other gram-negative and gram-positive bacterium, mycobacterium tuberculosis, Cryptococcal neoformans, or viral encephalitis; Upper and lower respiratory tract infections such as: bronchitis, bronchiolitis, pneumonia, otitis media, pharyngitis, sinusitis, epiglottitis and laryngitis; Infective endocarditis; Tuberculosis; Gasterointestinal infections such as: enterotoxigenic, enterhemorrhagic, and travelers diarrhea; pseudomembranous colitis; Shigellois; Salmonellosis; Campylobacteriosis; Yersiniosis; gasteroenteritis; Intra-abdominal infections of the stomach, biliary tract, proximal small-bowel, distal ileum, and colon; primary and secondary bacterial peritonitis; abscess; appendicitis; cholysystitis, cholangitis, contamination from abdominal trauma, pelvic inflammatory disease; Parasitic diseases, including protozoan infections, diseases from roundworms and flatworms (helminthiasis) and ectoparasites (pediculosis, acariasis), among others; Urinary tract infections, prostatitis, urethritis, epididymitis, cervicitis or vulvovaginitis, proctitis, salpingitis; Sepsis and septic shock; Superficial and invasive fungal infections such as: histoplasmosis, blastomycosis, coccidioidomycosis, cryptococcosis, Candidiasis, Apergillosis; Post-surgical infections; Degenerative joint diseases including osteoarthritis (degenerative join disease) as a result of trauma to the joint, infection of the joint, or age; Rheumatoid arthritis and psoriatic arthritis and/or autoimmune diseases in which the body attacks itself; Septic arthritis caused by joint infection; Gouty arthritis caused by deposition of uric acid crystals in the joint, causing inflammation; Bursitis inflammation of one or more bursae (small sacs) of synovial fluid in the body; Diseases resultant from vascular disease or injury from atherosclerosis, ischemia, or infarct including: stroke, cerebrovascular ischemia, cerebrovascular infarct, cerebrovascular accidents, myocardial ischemia and infarct; and Gasterointestinal inflammatory conditions such as peptic and duodenal ulcer disease, inflammatory bowel and Crohn's disease, toxic megacolon, colangeous colitis, lymphocytic colitis, ischemic colitis, diversion colitis, Behcet's colitis, inflammatory colitis.

“Diabetes” is generally considered a chronic disease that occurs when the pancreas does not produce enough insulin, or the body cannot effectively use the insulin it produces. One of the main functions of insulin is to lower blood glucose levels by enabling glucose to enter the cells of the body, where it is used for energy or stored for future use. Diabetes can refer to a disease diagnosed as diabetes according to the diagnostic standard, for example, of WHO (World Health Organization), Japan Diabetes Society, American Diabetes Association or European Association for the Study of Diabetes and includes Type 1 diabetes, Type 2 diabetes, gestational or pregnancy diabetes, and the like. Type 2 diabetes can be characterized by its resistance to the action of insulin, i.e., “insulin resistance.” Generally, a person who is insulin-sensitive requires a relatively small amount of insulin to maintain blood glucose levels in the normal range; however, a person who is insulin-resistant, may require significantly more insulin to achieve the same or similar blood-glucose-lowering effects.

“Insulin resistance” can mean a disease diagnosed as insulin resistance, based on the insulin resistance index (fasting blood sugar (mg/dL)xfasting insulin (microU/mL)÷405) or on the results obtained by examination by glucose clamp method or the like and includes syndrome X additionally. In addition to Type 2 diabetes, diseases with “insulin resistance” include, for example, fatty liver, particularly non-alcoholic fatty liver disease (NAFLD), non-alcoholic steatohepatitis (NASH), hyperglycemia, lipodosis, impaired glucose tolerance, hyperlipemia, pregnancy diabetes, polycystic ovary syndrome and the like.

Thus, a diabetes-related disorder can be hyperglycemia, hyperinsulinemia, impaired glucose tolerance, impaired fasting glucose, non-alcoholic fatty liver disease, dyslipidemia, hypertriglyceridemia, insulin resistance, and combinations thereof.

As used herein, a “genetic disease” or “genetic disorder” (wherein disease and disorder can be used interchangeably) refers to a condition caused by an abnormal genetic process. Examples of genetic disorders include, but are not limited to, cystic fibrosis, muscular dystrophies, color blindness, hemophilia, sickle-cell disease and genetic-related diseases such as cancer. In some embodiments, the genetic disorder is muscular dystrophy.

Cancers that have a genetic basis include cancers that are the result of genetically inherited mutations. Examples of such cancers include, but are not limited to, breast cancers, cancers which can be related to Li-Fraumeni syndrome, for example, childhood sarcomas, leukemias and brain cancers, cancers which can be related to Lynch syndrome, for example, colon cancers, bile duct cancers, brain cancers, endometrial cancers, kidney cancers, ovarian cancers, pancreatic cancers, small intestinal cancers, stomach cancers and ureter cancers, lung cancers, melanomas, prostate cancers, retinoblastomas, thyroid cancers and uterine cancers.

Moreover, cancers that have a genetic basis also include cancers that are the result of acquired mutations, for example, mutations resulting from diet, environment and/or lifestyle, or somatic mutations. Examples of such cancers include, but are not limited to, adrenal cancer, adrenal cortex cancer, bladder cancer, brain cancer, primary brain cancer, glioma, glioblastoma, breast cancer, cervical cancer, colon cancer (non-limiting examples include colorectal carcinomas such as colon adenocarcinoma and colon adenoma), endometrial cancer, epidermal cancer, esophageal cancer, gall bladder cancer, genitourinary cancer, head or neck cancer, kidney cancer, liver cancer, lung cancer (non-limiting examples include adenocarcinoma, small cell lung cancer and non-small cell lung cancer), lymphomas (non-limiting examples include B-cell lymphoma, T-cell lymphoma, Hodgkin's lymphoma, non-Hodgkin's lymphoma), melanoma, malignant melanoma, malignant carcinoid carcinoma, malignant pancreatic insulinoma, myeloma, multiple myeloma, ovarian cancer, pancreatic cancer (such as exocrine pancreatic carcinoma), prostate cancer, renal cell cancer, skin cancer, such as, in addition to others previously mentioned, squamous cell carcinoma, stomach cancer, testicular cancer, thyroid cancer, thyroid follicular cancer, Wilms' tumor, choriocarcinoma, mycosis fungoides, malignant hypercalcemia, cervical hyperplasia, leukemia, acute lymphocytic leukemia, chronic lymphocytic leukemia, hairy cell lymphoma, Burkett's lymphoma, acute myelogenous leukemia, chronic myelogenous leukemia, myelodysplastic syndrome, promyelocytic leukemia, acute granulocytic leukemia, chronic granulocytic leukemia, fibrosarcoma, rhabdomyosarcoma, astrocytoma, neuroblastoma, schwannoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, seminoma, teratocarcinoma, xenoderoma pigmentoum, keratoctanthoma and retinoblastoma.

A diagnosis of any of these diseases may be made by clinical observation and assessment and/or through diagnostic testing recognized as acceptable by those skilled in the art for determining the amount and/or duration of therapy.

A “subject” of the invention includes any animal having or susceptible to the conditions described herein for which prevention or treatment of said condition is needed and/or desired. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. Subjects of the invention can be a subject known or believed to be at risk for the condition for which prevention or treatment is needed and/or desired. Alternatively, a subject according to the invention can also include a subject not previously known or suspected to be at risk for the condition for which prevention or treatment is needed or desired. As a further option, the subject can be a laboratory animal and/or an animal model of disease. Suitable subjects include both males and females and subjects of any age, including embryonic (e.g., in utero or in ovo), infant, juvenile, adolescent, adult and geriatric subjects. The subject may also be of any race or ethnicity, including, but not limited to, Caucasian, African-American, African, Asian, Hispanic, Indian, etc., and combined backgrounds.

A “subject in need thereof” in the context of therapy, is a subject known to be, or suspected of having or being at risk of having a disease or disorder, and may benefit from said therapy, i.e. is in need thereof.

Embodiments of the present invention further provide a transformed cell produced by the process of delivering a nucleic acid and/or a vector to a cell, the process comprising, consisting essentially of, or consisting of administering the nucleic acid and/or vector in combination with at least one drug that modulates a membrane transport mechanism. As used herein, a “transformed” cell is a cell that has been transformed, transduced and/or transfected with the optimized polynucleotide provided by the nucleic acid and/or a vector.

The cell(s) into which the nucleic acid and/or vector can be introduced may be of any type, including but not limited to neural cells (including cells of the peripheral and central nervous systems, in particular, brain cells such as neurons, oligodendrocytes, glial cells, astrocytes), lung cells, cells of the eye (including retinal cells, retinal pigment epithelium, and corneal cells), epithelial cells (e.g., gut and respiratory epithelial cells), skeletal muscle cells (including myoblasts, myotubes and myofibers), diaphragm muscle cells, dendritic cells, pancreatic cells (including islet cells), hepatic cells, a cell of the gastrointestinal tract (including smooth muscle cells, epithelial cells), heart cells (including cardiomyocytes), bone cells (e.g., bone marrow stem cells), hematopoietic stem cells, spleen cells, keratinocytes, fibroblasts, endothelial cells, prostate cells, joint cells (including, e.g., cartilage, meniscus, synovium and bone marrow), germ cells, and the like. Alternatively, the cell may be any progenitor cell. As a further alternative, the cell can be a stem cell (e.g., neural stem cell, liver stem cell). As still a further alternative, the cell may be a cancer or tumor cell (cancers and tumors are described above). Moreover, the cells can be from any species of origin, as indicated above. Such cells can be isolated and/or present in an animal, e.g., a transgenic animal.

Accordingly, another embodiment of the present invention relates to a transgenic animal comprising the polynucleotide, vector, and/or transformed cell of the invention. A transgenic animal may include, but is not limited to, a farm animal (e.g., pig, goat, sheep, cow, horse, rabbit and the like), rodents (such as mice, rats and guinea pigs), and domestic pets (for example, cats and dogs). In some embodiments, the transgenic animal is not a human.

A transgenic animal may be produced by methods well understood by those or ordinary skill in the art. Briefly, nucleic acid molecules can be introduced into embryos by a variety of means including but not limited to microinjection, calcium phosphate mediated precipitation, liposome fusion, or retroviral infection of totipotent or pluripotent stem cells. The transformed cells can then be introduced into embryos and incorporated therein to form transgenic animals. Methods of making transgenic animals are described, for example, in Transgenic Animal Generation and Use by L. M. Houdebine, Harwood Academic Press, 1997. Transgenic animals also can be generated using methods of nuclear transfer or cloning using embryonic or adult cell lines as described for example in Campbell et al., Nature 380:64-66 (1996) and Wilmut et al., Nature 385:810-813 (1997). Further a technique utilizing cytoplasmic injection of DNA can be used as described in U.S. Pat. No. 5,523,222.

The vectors may be introduced to cells in vitro for the purpose of administering/delivering the modified cell to a subject. In particular embodiments, the cells have been removed from a subject, the vector is introduced therein, and the cells are then replaced back into the subject. Methods of removing cells from subject for treatment ex vivo, followed by introduction back into the subject are known in the art (see, e.g., U.S. Pat. No. 5,399,346). Alternatively, the recombinant vector is introduced into cells from another subject, into cultured cells, or into cells from any other suitable source, and the cells are administered to a subject in need thereof.

Suitable cells for ex vivo gene therapy are as described above. Dosages of the cells to administer to a subject will vary upon the age, condition and species of the subject, the type of cell, the nucleic acid being expressed by the cell, the mode of administration, and the like. Typically, at least about 10² to about 10⁸ or about 10³ to about 10⁶ cells will be administered per dose in a pharmaceutically acceptable carrier. In particular embodiments, the cells transduced with the vector are administered to the subject in an effective amount and may be in combination with a pharmaceutical carrier.

A further aspect of the invention is a method of administering the vectors and drug of the invention to subjects. In particular embodiments, the method comprises, consists essentially of or consists of a method of delivering a polynucleotide of interest to an animal subject, the method comprising, consisting essentially of, or consisting of: administering an effective amount of a vector according to the invention to an animal subject. Administration of the vectors of the present invention to a human subject or an animal in need thereof can be by any means known in the art. Optionally, the vector is delivered in an effective dose in a pharmaceutically acceptable carrier.

An effective amount of a composition of this invention will vary from composition to composition and subject to subject, and will depend upon a variety of factors such as age, species, gender, weight, overall condition of the subject and the particular disease or disorder to be treated. An effective amount can be determined in accordance with routine pharmacological procedures know to those of ordinary skill in the art. As the skilled artisan would understand, the specific dose would depend on the size of the target/subject and the nature of the target/subject.

The frequency of administration of a composition of this invention can be as frequent as necessary to impart the desired therapeutic effect. For example, the composition can be administered one, two, or more times per day, one, two, three, four or more times a week, one, two, three, four or more times a month, one, two, three or four times a year and/or as necessary to control a particular condition and/or to achieve a particular effect and/or benefit. In particular embodiments, more than one administration (e.g., two, three, four or more administrations) may be employed to achieve the desired level of gene expression over a period of various intervals, e.g., daily, weekly, monthly, yearly, etc. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

Having described the present invention, the same will be explained in greater detail in the following examples, which are included herein for illustration purposes only, and which are not intended to be limiting to the invention.

### EXAMPLES

Fatty acid/glucose transporter-activating drugs such as insulin, insulin secretion activators, such as repaglidine and sulfonylureas or any of the glucose transporter activator, or compounds able to activate the fatty acid transporters will be used for the delivery of following nucleic acid cargos including nucleotides, oligonucleotides and long chain nucleotides (up to 200 base pairs) and expression vectors (such as plasmid or synthetic expression sequences).

The nucleotides, oligonucleotides and long chain nucleotides can be of any chemical modification, such as 2′-O-methyl phosphorothioate, 2′-O-methoxyethyl (MOE), Locked/bridged nucleic acids (LNA/BNA), Phosphoramidate, 2′-O-aminopropyl phosphodiester ribonucleotides, Phosphorodiamidate Morpholino Oligonucleotides (PMO), Peptide nucleic acid (PNA), Hexitol nucleic acid (HNA) and tricyclo-DNA (tcDNA), with and without further modification.

This invention can also be applied for the delivery of plasmid or other expression vectors with and without any modification for any gene therapy purpose. The plasmid or other expression vectors can be modified to contain fatty acid or sugar for delivery enhancement.

Nucleic acid cargos can be directly modified with fatty acid or sugar for further delivery enhancement. The nucleic acid cargos can be delivered parenterally such as intraperotoneally (i.p.), intravenously (i.v.), subcutaneously (SC), intramuscularly (i.m.), or directly into targeted tissues. Fatty acids, such as medium, long chain and very long chain fatty acids, can also be directly conjugated or bound to the oligonucleotide and gene expression vector sequences for enhanced delivery.

A combination of drugs activating the fatty acid glucose/transportation pathways with the oligonucleotide/gene vector (nucleic acid cargo) modified with fatty acids may also be employed.

The fatty acid/glucose transporter-activating drugs are administrated orally, i.p., i.v., i.m. and s.c. directly into target tissues prior to (5 hours to 1 minute), at the same time (simultaneously in mixture or separately with nucleotides or gene/expression vector), or after (up to 2 hours) the delivery of cargo nucleic acids. The two components, transporter activating drugs and cargo nucleic acids, can also be administrated in combination with any other agent by any route of administration with any frequency.

With the significantly enhanced delivery of nucleotides and expression vectors, a therapeutic effect for many diseases can be achieved locally and systemically.

### Example 1

### The mdx Dystrophic Mice Containing a Nonsense Mutation in the Dystrophin Exon 23 are Treated with Antisense Oligonuleotide

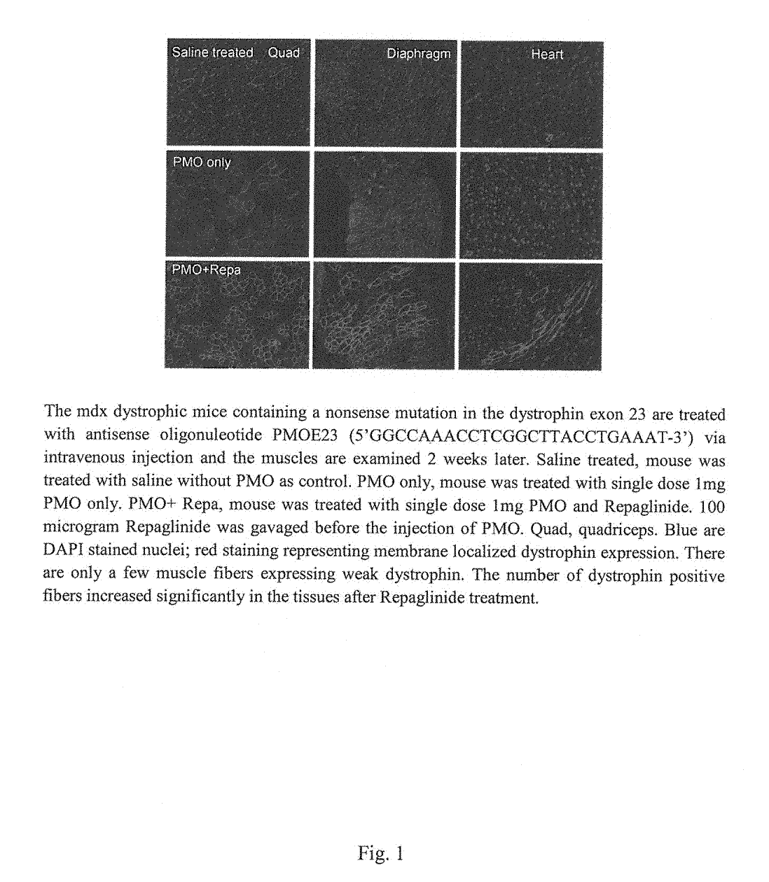

The mdx dystrophic mouse contains a nonsense point mutation in the dystrophin exon 23. PMOE23 (5′GGCCAAACCTCGGCTTACCTGAAAT-3′) (SEQ ID NO:1) oligo binds to the exon-intron boundary of the dystrophin exon 23, resulting in the skip of the exon 23 and restoration of the reading frame and the expression of the dystrophin protein. The mdx mice are treated with antisense oligonuleotide PMOE23 (5′GGCCAAACCTCGGCTTACCTGAAAT-3′) (SEQ ID NO:1) via intravenous injection and the muscles are examined 2 weeks later. Saline treated, mouse was treated with saline without PMO as control. PMO only, mouse was treated with single dose 1 mg PMO only. PMO+Repa, mouse was treated with single dose 1 mg PMO and Repaglinide. 100 microgram Repaglinide was gavaged before the injection of PMO. Quad, quadriceps. Blue are DAPI stained nuclei; red staining representing membrane localized dystrophin expression. There are only a few muscle fibers expressing weak dystrophin. The number of dystrophin positive fibers increased significantly in the muscle tissues of the mice treated with same amount of PMO in combination with Repaglinide treatment. See FIG. 1.

### Example 2

### A Comparison of Levels of Dystrophin Expression after Treatment with a Morpholino Antisense Oligonucleotide

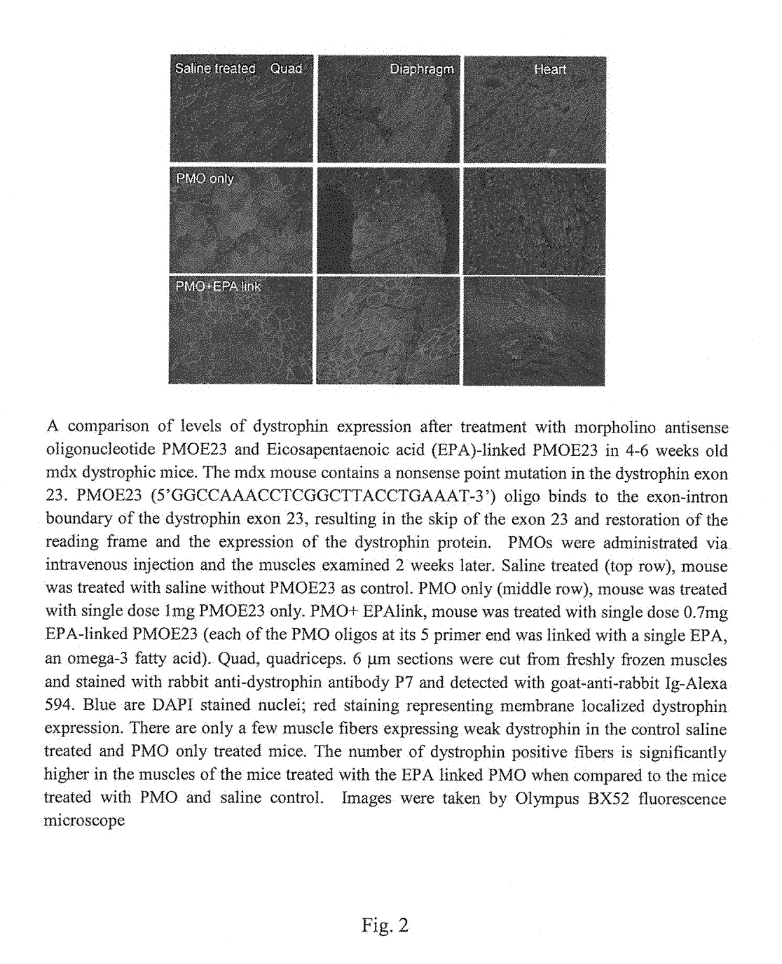

A comparison of levels of dystrophin expression after treatment with morpholino antisense oligonucleotide PMOE23 and Eicosapentaenoic acid (EPA)-linked PMOE23 in 4-6 weeks old mdx dystrophic mice. The mdx mouse contains a nonsense point mutation in the dystrophin exon 23. PMOE23 (5′GGCCAAACCTCGGCTTACCTGAAAT-3′) (SEQ ID NO:1) oligo binds to the exon-intron boundary of the dystrophin exon 23, resulting in the skip of the exon 23 and restoration of the reading frame and the expression of the dystrophin protein. PMOs were administrated via intravenous injection and the muscles examined 2 weeks later. Saline treated (top row), mouse was treated with saline without PMOE23 as control. PMO only (middle row), mouse was treated with single dose 1 mg PMOE23 only. PMO+EPAlink, mouse was treated with single dose 0.7 mg EPA-linked PMOE23 (each of the PMO oligos at its 5 primer end was linked with a single EPA, an omega-3 fatty acid). Quad, quadriceps. 6 μm sections were cut from freshly frozen muscles and stained with rabbit anti-dystrophin antibody P7 and detected with goat-anti-rabbit Ig-Alexa 594. Blue are DAPI stained nuclei; red staining representing membrane localized dystrophin expression. There are only a few muscle fibers expressing weak dystrophin in the control saline treated and PMO only treated mice. The number of dystrophin positive fibers is significantly higher in the muscles of the mice treated with the EPA linked PMO when compared to the mice treated with PMO and saline control. Images were taken by Olympus BX52 fluorescence microscope. See FIG. 2.

### Example 3

### A Comparison of Levels of Dystrophin Expression after Treatment with Morpholino Antisense Oligonucleotides in Combination with the Use of Insulin

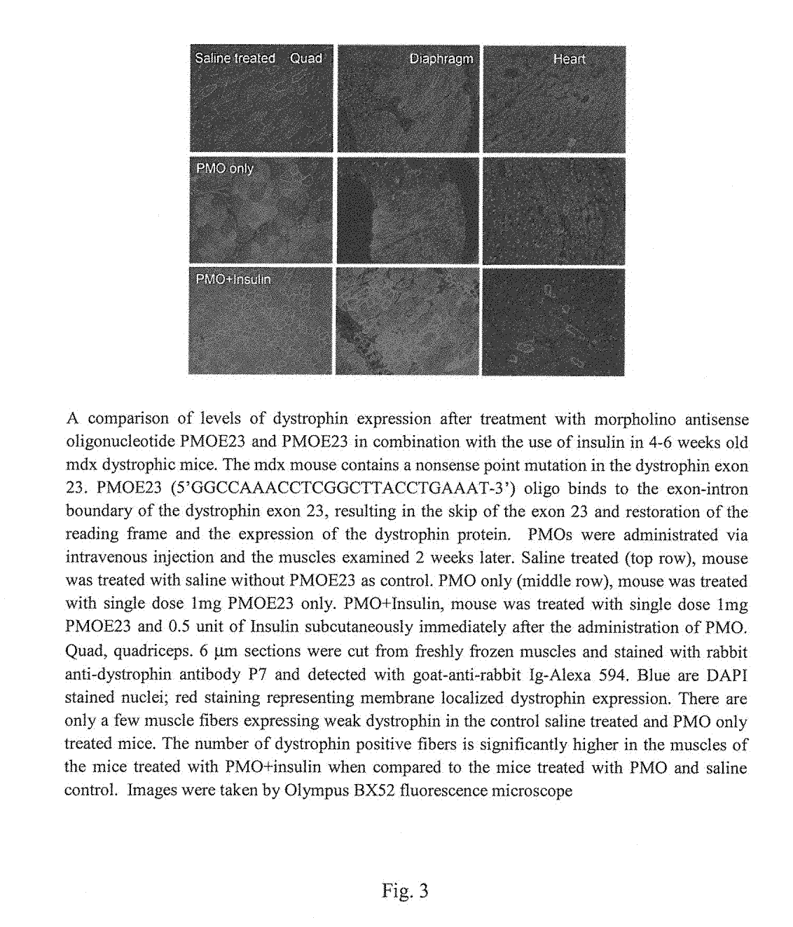

A comparison of levels of dystrophin expression after treatment with morpholino antisense oligonucleotide PMOE23 and PMOE23 in combination with the use of insulin in 4-6 weeks old mdx dystrophic mice. The mdx mouse contains a nonsense point mutation in the dystrophin exon 23. PMOE23 (5′GGCCAAACCTCGGCTTACCTGAAAT-3′) (SEQ ID NO:1) oligo binds to the exon-intron boundary of the dystrophin exon 23, resulting in the skip of the exon 23 and restoration of the reading frame and the expression of the dystrophin protein. PMOs were administrated via intravenous injection and the muscles examined 2 weeks later. Saline treated (top row), mouse was treated with saline without PMOE23 as control. PMO only (middle row), mouse was treated with single dose 1 mg PMOE23 (in saline) only. PMO+Insulin, mouse was treated with single dose 1 mg PMOE23 and 0.5 unit of Insulin subcutaneously immediately after the administration of PMO. Quad, quadriceps. 6 μm sections were cut from freshly frozen muscles and stained with rabbit anti-dystrophin antibody P7 and detected with goat-anti-rabbit Ig-Alexa 594. Blue are DAPI stained nuclei; red staining representing membrane localized dystrophin expression. There are only a few muscle fibers expressing weak dystrophin in the control saline plus PMO only treated mice. The number of dystrophin positive fibers is significantly higher in the muscles of the mice treated with PMO+insulin when compared to the mice treated with PMO and saline control. Images were taken by Olympus BX52 fluorescence microscope. See FIG. 3.

It will be understood by those of skill in the art that numerous and various modifications can be made without departing from the spirit of the present invention. Therefore, it should be clearly understood that the forms of the present invention are illustrative only and are not intended to limit the scope of the present invention.

All publications, patent applications, patents, patent publications, sequences identified by GenBank® database accession numbers and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

What is claimed is:

1. A method of delivering a nucleic acid to a cell or tissue, the method comprising administering the nucleic acid in combination with at least one drug that modulates a membrane transport mechanism.

2. The method of claim 1, wherein the membrane transport mechanism is modulated by insulin and/or by the release of insulin.

3. The method of claim 1, wherein, the nucleic acid is an oligonucleotide or a long chain oligonucleotide.

4. The method of claim 1, wherein the nucleic acid is selected from the group consisting of an anti sense oligonucleotide, an siRNA, a gapmer, and a microRNA.

5. The method of claim 1, wherein the nucleic acid is an antisense oligonucleotide.

6. The method of claim 1, wherein the nucleic acid is chemically modified.

7. The method of claim 6, wherein the chemical modification is selected from the group consisting of a2′-O-methyl phosphothioate modification, a 2′-O-methoxyethyl (MOE) modification, a locked nucleic acid (LNA) modification, a bridged nucleic acid (BNA) modification, a phosphoramidate modification, a 2′-O-aminopropyl phosphodiester ribonucleotide modification, a phosphodiamidate morpholine oligonucleotide (PMO) modification, a peptide nucleic acid (PNA) modification, a, hexitol nucleic acid (HNA) modification, and a tricyclo-DNA (tcDNA) modification, or, a combination of any thereof.

8. The method of claim 6, wherein the chemical modification is a PMO modification.

9. The method of claim 1, wherein the membrane transport mechanism is a fatty acid transport system.

10. The method of claim 1, wherein the membrane transport mechanism is a glucose transport system.

11. The method of claim 1, wherein the drug is at least one selected from the group consisting of a sulfonylurea, a biguanide, a meglitinide, a thiazolidinedione, a DPP-4 modulator, a SGLT2 modulator, an alpha-glucosidase modulator, a bile acid sequestrant, insulin, dipyridamole, oligomycin and a K⁺_{ATP} channel modulator.

12. The method of claim 1, wherein the drug is insulin and/or repaglidine.

13. The method of claim 1, wherein a fatty acid moiety or a sugar moiety is linked to the nucleic acid.

14. The method of claim 13, wherein the fatty acid is a medium chain, long chain or very long chain fatty acid.

15. The method of claim 1, wherein the nucleic acid is administered before the drug, after the drug, or the same time as the drug.

16. The method of claim 15, wherein the nucleic acid is administered in a composition including the drug.

17. The method of claim 15, wherein the nucleic acid is administered orally or parenterally.

18. The method of claim 15, wherein the nucleic acid and drug are administered intraperitoneally (i.p.), intravenously (i.v.), subcutaneously (SC), intramuscularly (i.m.), or directly into target tissues.

19. The method of claim 15, wherein the nucleic acid and drug are administered at the same site.

20. The method of claim 15, wherein the nucleic acid and drug are administered at different sites.

21. A method of treating cancer, an infectious disease, diabetes or a genetic disorder, the method comprising administering to a subject in need thereof a nucleic acid in combination with at least one drug that modulates a membrane transport mechanism.

22. The method of claim 21, wherein the membrane transport mechanism is modulated by insulin and/or by the release of insulin.

23. The method of claim 21, wherein, the nucleic acid is an oligonucleotide or a long chain oligonucleotide.

24. The method of claim 21, wherein the nucleic acid is selected from the group consisting of an antisense oligonucleotide, an siRNA, a gapmer, and a microRNA.

25. The method of claim 21, wherein the nucleic acid is an antisense oligonucleotide.

26. The method of claim 21, wherein the nucleic acid is chemically modified.

27. The method of claim 26, wherein the chemical modification is selected from the group consisting of a2′-O-methyl phosphothioate modification, a 2′-O-methoxyethyl (MOE) modification, a locked nucleic acid (LNA) modification, a bridged nucleic acid (BNA) modification, a phosphoramidate modification, a 2′-O-aminopropyl phosphodiester ribonucleotide modification, a phosphodiamidate morpholine oligonucleotide (PMO) modification, a peptide nucleic acid (PNA) modification, a, hexitol nucleic acid (RNA) modification, and a tricyclo-DNA (tcDNA) modification, or a combination of any thereof.
